(19) United States
(12) Patent Application Publication
(10) Pub. No.: US 2008/0236598 A1
(43) Pub. Date: Oct. 2, 2008

(54) Drape for open tracheal suctioning

(76) Inventor: Fred Gobel, Wilhelmsfeld (DE)

Correspondence Address: KIMBERLY-CLARK WORLDWIDE, INC.;Catherine E. Wolf, 401 NORTH LAKE STREET, NEENAH, WI, 54956, US

(21) Appl. No.: 11/731,068

(22) Filed: Mar. 30, 2007

(51) Int. Cl. A61B 19/08

(52) U.S. Cl. 128/849

(57) ABSTRACT

A surgical drape that promotes enhanced protection of the surgical site from pathogens is provided. The surgical drape includes a base sheet and a connecting element in communication with the drape, the connecting element being adapted to enable a male or female fit into a connector of a medical device and further adapted to occlude an opening in the central region of the base sheet.

### BACKGROUND

Various types of surgical drapes are known and used to keep a surgical site on a patient sterile during a surgical procedure. Traditionally, surgical drapes were linen or woven cloth, and were sterilized after each use for reuse. More recently, disposable sterile drapes have been introduced, in which a nonwoven paper or fabric forms a substantial part of the drape. A reinforcement area is often placed around a fenestration or an edge of disposable surgical drapes to provide structural strength and to absorb bodily fluids from the surgical site. Many disposable drapes also include a number of layers of different materials for the drape area and reinforcement area, with each layer providing a different property to the drape. For example, spunbond fabrics, meltblown fabrics, and polymer films have been used as layers in disposable drapes.

Conventionally, surgical drapes are square shaped (although they may be any shape) and have a cut from one edge towards a more central region of the drape, the more central region having an opening. In certain medical procedures involving tubes such as tracheal airway opening, tracheal suctioning, or bronchoscopy, the shaft of the tube extends out beyond the patient's face level leaving the tube exposed to potential pathogens and contamination.

For example, during conventional tracheal suctioning, the inserted tip of the suctioning catheter may unintentionally make contact with the unprotected tube shaft or connector because the tube shaft extends beyond the patient's face level and is not covered by the drape. Thus, a risk of accidental inoculation of pathogens into the lower airway is possible.

Additionally, for example, during open tracheal suctioning it may be necessary for the medical professional to manipulate the position of the tracheal tube manually, which requires physical contact with the outside of the tube shaft which could contaminate the manipulating hand or glove with pathogens found on the outside of the tube shaft.

Thus, there remains a need for protection of the tube shaft from pathogens during medical procedures involving tubes which extend outside of the patient's body.

### SUMMARY OF INVENTION

The present invention provides for a drape for covering a patient during a medical procedure. The drape includes a base sheet having an upper surface, a lower surface, a plurality of edges, and a central region defining an opening, the opening adapted to be positioned over the medical procedure site. The drape also includes a connecting element adapted to enable a male or female fit into a connector of a medical device, whereby the connecting element is in communication with the drape and is further adapted to occlude the opening.

Desirably, the connecting element may partially extend above the upper surface of the central region and partially extend below the lower surface of the central region. The base sheet and/or central region of the base sheet may comprise a layer of liquid absorbent material or a barrier fabric or combinations of both. The liquid absorbent material or barrier fabric may include antiseptics on their surface layers or may include antiseptics within the absorbent material or barrier fabric themselves.

Desirably, the central region of the base sheet defining the opening is adapted to surround the medical device upon insertion of the connecting element into the connector of the medical device and is further adapted to allow manipulation of the medical device without touching of the medical device. The central region of the drape defining the opening may include a plastic film which may be transparent. Additionally, the connecting element may include an outside male port or female port which extends beyond the upper surface of the base sheet and has a diameter between about 10 and about 20 mm.

The medical device may be an endotracheal tube, a tracheostomy tube, bronchoscope, vascular catheter, urological catheter, or any medical device that extends outward from the body during at least a portion of a medical procedure.

The drape may be pre-folded so that the connecting element is available for connection with the connector. Additionally, the drape may include a tray in communication with the base sheet.

The central region of the base sheet defining the opening may be further defined by a central duct when the drape is pre-folded, the duct being adapted to receive the medical device and the connector.

Another aspect of the invention addresses a method of providing a sterile field about a patient during a medical procedure. The method includes providing a drape which includes a base sheet having a connecting element which occludes an opening in the base sheet, the base sheet having an upper surface, a lower surface, and a plurality of edges; positioning the opening over a medical procedure site; connecting a connecting element into the connector of a medical device; and manipulating the medical device without touching the medical device.

Yet another aspect of the invention addresses a drape adapted for covering a patient during a tracheal, bronchoscopic, vascular, or urological procedure. The drape includes a base sheet having an upper surface, a lower surface, a plurality of edges, and a central region defining an opening, the opening adapted to be positioned over the tracheal, vascular, bronchoscopic, or urological procedure site. The drape also includes a connecting element adapted to enable a male or female fit into a connector of a medical device, whereby the connecting element is in communication with the drape and is further adapted to occlude the opening.

### BRIEF DESCRIPTION OF DRAWINGS

FIG. 1 is a view of a surgical drape covering a patient having an inserted endotracheal tube.

FIG. 2 is a cross-sectional view of the central region of the base sheet, the opening of the central region, and its integrated connecting element having an optional outside female port.

FIG. 3 is a cross-sectional view of the central region of the base sheet, the opening of the central region, and its integrated connecting element having an optional male outside connector.

FIG. 4 is a cross-sectional view of the central duct which defines the central region of the base sheet.

FIG. 5 is a cross-sectional view of the corner end of the folded surgical drape.

FIG. 6 is a view of a package enclosing the surgical drape, the package having perforations at the ends.

FIG. 7 is a view of a view of a surgical drape incorporating a tray and covering a patient having an inserted endotracheal tube.

### DEFINITIONS

As used herein, the terms “germicidal agent” or “germicidal agents” refer to chemicals or other substances that either kill or slow the growth of microbes. Among the germicidal agents in use today are antibacterial agents (which kill bacteria), antiviral agents (which kill viruses), antifungal agents (which kill fungi), and antiparasitic drugs (which kill parasites). A main category of germicidal agents are surface disinfectants, otherwise known as “biocides.”

The term “biocides” is a general term describing a chemical agent, such as a pesticide, usually broad spectrum, which inactivates living microorganisms. Because biocides range in germicidal activity, other terms may be more specific, including “-static,” referring to agents that inhibit growth (e.g., bacteriostatic, fungistatic, or sporistatic) and “-cidal,” referring to agents that kill the target organism (e.g., bactericidal, fungicidal, sporicidal, or virucidal). Biocides have multiple targets and modes of action, which for instance, may include physical disruption and permanent damage to the outer cell membrane of a bacterial microbe. Some example of useful biocide chemistries include biguanides (e.g., chlorohexidine, alexidine, polyhexamethylene biguanide, and relevant salts thereof), halogen-releasing agents (e.g.: iodine, iodophors, sodium hypochlorite, N-halamine, etc.), stabilized oxidants such as chlorine dioxide, stabilized peroxide (e.g., urea peroxide, mannitol peroxide) metal-containing species and oxides thereof (e.g.: silver, copper, selenium, etc. either in particle form or incorporated into a support matrix such as a zeolite or polymer), sulfides (e.g., sodium metabisulfite), bis-phenols (e.g., triclosan, hexachlorophene, etc), quaternary ammonium compounds (e.g., benzalkonium chloride, cetrimide, cetylpyridium chloride, quaternized cellulose and other quaternized polymers, etc.), various “naturally occurring” agents (e.g., polyphenols from green or black tea extract, citric acid, chitosan, anatase TiO₂, tourmaline, bamboo extract, neem oil, etc.), hydrotropes (e.g., strong emulsifiers) and chaotropic agents (e.g., alkyl polyglycosides) and synergistic combinations thereof. Depending on substrate chemistry (polyolefin vs. cellulosic-based materials) and the method of incorporation into the product (topical vs. grafting), many of the above chemistries could be used alone or in concert to achieve the final claimed product properties of interest.

### DETAILED DESCRIPTION

The drapes of the present invention are designed for decrease the introduction of pathogens into the area surrounding the surgical site. Specifically, the drapes of the present invention are designed to prevent pathogens present on surgical or medical procedure instruments from entering the surrounding surgical or medical procedure site when the medical or surgical device extends outward from the body during at least a portion of the surgical or medical procedure. The prevention of the transmission of pathogens is accomplished by covering the instrument with drape material so that the instrument is not exposed to the outside atmosphere after insertion of the instrument into a connecting element.

Additionally, the prevention of the transmission of pathogens is accomplished by preventing the medical professional from directly touching the surgical or medical procedure instrument during the medical or surgical procedure. In this regard, the medical professional may manipulate the instrument (by touching the drape material but not the instrument itself during a medical procedure. Thus, the use of the drape significantly lowers the risk of passing pathogens onto the surgical instrument or having pathogens from the surgical instrument passed onto the gloves of the medical professional. This significantly decreases the chance of exogenous infection entering the patient's body through the surgical or medical procedure site.

Surgical drapes formed in accordance with the present invention can generally possess any of a variety of sizes and shapes, depending on the particular use of the drape and its desired properties. For example, certain surgical drape configurations are described in U.S. Pat. No. 6,055,987, which is incorporated herein by reference for all purposes.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

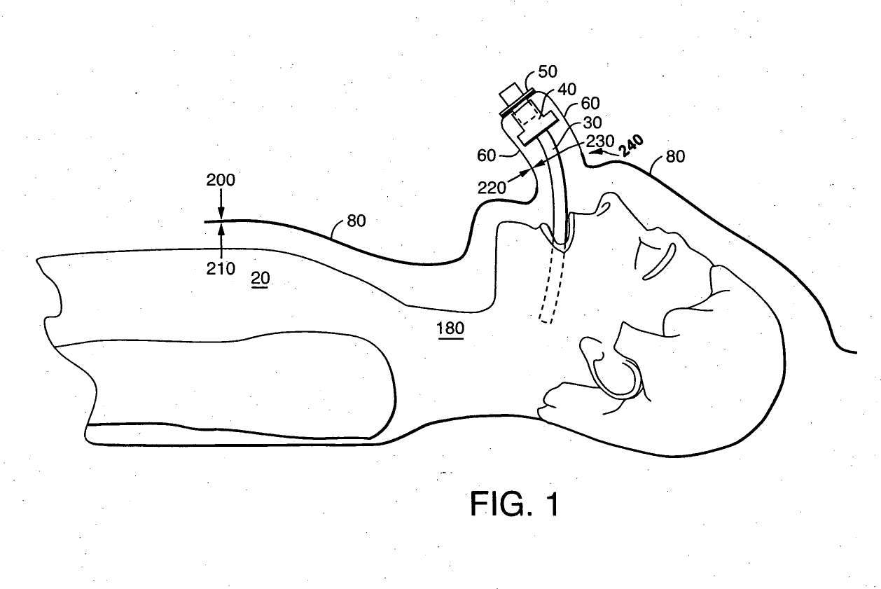

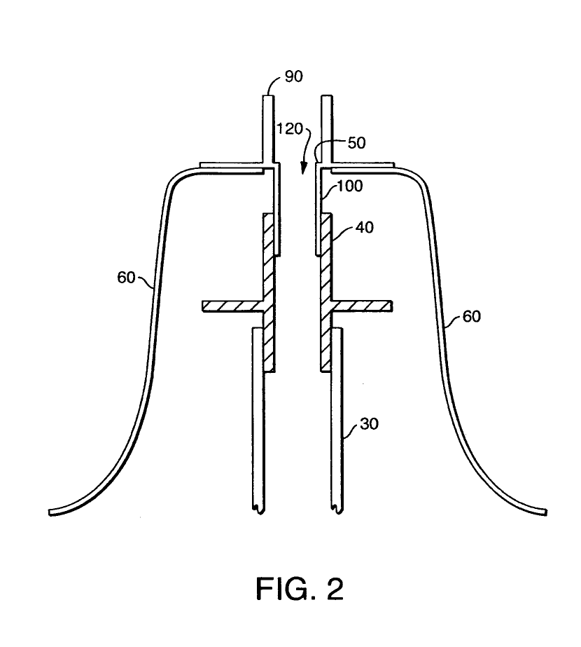

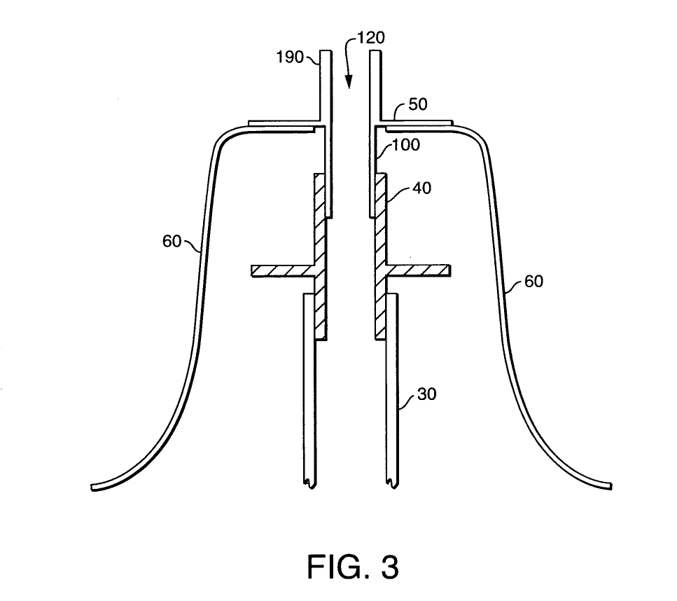

Referring to FIGS. 1-3 (FIGS. 2 and 3 are cross-sectional views), a surgical drape having a base sheet 80 and central region 60 is illustrated. The drape may cover the body in its entirety including the chest 20 and neck 180 regions or may partially cover the body. The base sheet 80 of the drape includes an upper surface 200 (away from the patient) and a lower surface 210 (towards the patient). The sheet 80 may be formed from one or more materials, for example one or more nonwoven layers, adhesive layers, film layers, and so forth. The base sheet 80 may be hydrophilic or hydrophobic or may have portions with each characteristic, and may be chemically treated to achieve desired water absorbency properties. Desirably, the base sheet 80 may be a nonwoven surface layer joined to a barrier layer by an adhesive layer such as, for example, a meltblown adhesive layer. The nonwoven layer may be a spunbond polyolefin (i.e. propylene) material sheet with a basis weight of about 5 to about 50 gsm, more desirably about 10 to about 30 gsm, and still more desirably about 20 gsm, and having been bonded by a repeating pattern of discretely fused spaced apart bonds or other conventional bonding methods. The meltblown adhesive layer may be made from an amorphous polyolefin applied to the spunbond material at a rate of, for example, from about 0.5 to 5 gsm, more desirably about 1 to about 4 gsm, and still more desirably about 3 gsm. The barrier layer may be, for example, a polyethylene and calcium carbonate film of from about 0.5 to about 3 mils thick, more desirably about 1.5 mils thick. The film may be stretched in one direction prior to lamination to the spunbond by the meltblown adhesive layer. The film provides an impermeable barrier to aqueous fluids and alcohol solutions; the inclusion of the calcium carbonate and subsequent stretching improving moisture vapor permeability. It is contemplated that the nonwoven layer may be the surface of the base sheet or the meltblown adhesive layer may be the surface of the base sheet.

The base sheet may incorporate anti-microbial materials. Although any conventional method of incorporating anti-microbial materials may be used, the method may include melt addition of the active agent to a polymer melt during extrusion and spinning of fibers and manufacturing of nonwoven materials used in making products; topical application methods that may or may not impart “sidedness” to the fabrics used in constructing the finished products; and other non-standard methods such as plasma treatment, electrostatic attachment, radiation surface graft copolymerizations using for example UV, gamma rays and electron-beam radiation sources, or the use of chemical initiation to produce graft copolymerized surfaces having anti-microbial activity, etc.

These anti-microbial materials include, but are not limited to germicidal agents and biocides. Desirably, the use of anti-microbial materials in the base sheet will destroy or neutralize a broad spectrum of microorganisms including at a minimum Gram positive and Gram negative bacteria, including resistant strains thereof, for example methicillan-resistant Staphylococcus aureus (MRSA), vancomycin-resistant Enterococci (VRE) and penicillin-resistant Streptococcus pneumoniae (PRSP) strains. Desirably, they will destroy or neutralize all bacteria (Gram+, Gram− and acid fast strains) and yeasts such as Candida albicans. Even more desirably, this will include all bacteria (Gram+, Gram−, and acid fast), yeasts, and both envelope and naked viruses such as human influenza, rhinovirus, poliovirus, adenovirus, hepatitis, HIV, herpes simplex, SARS, and avian flu. This should result in fewer viable pathogens on the treated surface as compared to an untreated surface, and prevent or minimize the contact transfer of pathogens.

The germicidal compositions utilized may be one or more germicidal reagents. These reagents may be effective by themselves or may be combined to produce a synergistic effect that is non-additive of the individual components. These germicidal reagents may be further combined with processing aids and/or other ingredients that provide functional properties to the compositions. Exemplary germicidal compositions may be based on cationic polymers, such as quaternary ammonium compounds and polymeric biguanides, alcohols, and surfactants. Combinations of cationic polymers such as quaternary ammonium compounds (e.g., quaternary ammonium cellulose and quaternary ammonium Sloane), polymeric biguanides, surfactants, alcohols, and organic acids, such as acetic, citric, benzoic acids, may produce non-additive, synergistic systems with broad pathogen efficacy. The combinations with other germicidal compounds, surfactants, appear to improve germicidal efficacy of polymeric biguanides over treatments with that employ polymer biguanides alone. Although not required, it is desirable that the germicidal compounds be odorless to humans; that is, the composition should desirably be undetectable to the human olfactory system.

Poly-hexamethylene biguanide (PHMB) hydrochloride is an exemplary cationic biguanide that is useful for providing germicidal surface-covering assemblies.

Commercially available versions of PHMB, such as under the trade names Cosmocil CQ (20 wt. % PHMB in water) or Vantocil, a heterodisperse mixture of PHMB with a molecular weight of approximately 3,000, are active against gram-positive and gram-negative bacteria, but may not be sporicidal.

Additional active germicidal agents may include a quaternary ammonium compound, a quaternary ammonium siloxane, a polyquaternary amine; metal-containing species and oxides thereof, either in particle form or incorporated into a support matrix or polymer; halogens, a halogen-releasing agent or halogen-containing polymer, a bromo-compound, a chlorine dioxide, a thiazole, a thiocynate, an isothiazolin, a cyanobutane, a dithiocarbamate, a thione, a triclosan, an alkylsulfosuccinate, an alkyl-amino-alkyl glycine, a dialkyl-dimethyl-phosphonium salt, a cetrimide, hydrogen peroxide, 1-alkyl-1,5-diazapentane, or cetyl pyridinium chloride.

Table 1 summarizes various biocides and processing aids that may be used in germicidal compositions that may be incorporated in the base sheet. It also lists their common chemical names or commercial names. Quaternary ammonium compounds, such as commercially available under the names of Aegis™ AEM 5700 (Dow Corning, Midland, Mich.) and Crodacel QM (Croda, Inc., Parsippany, N.J.), with certain surfactants such as alkyl-polyglycosides, available commercially under the name Glucopon 220 UP (Cognis Corp, Ambler, Pa.), and chitosan glycolate, available under the name Hydagen CMF and Hydagen HCMF (Cognis Corp., Cincinnati, Ohio), can significantly enhance the killing efficacy of PHMB in a synergistic fashion as will be demonstrated in the tables herein. One should note that many of the biocides described herein may be used singly or in combination in a variety of products which vary considerably in activity against microorganisms.

TABLE 1
Table of Active Reagents and Processing Aids

| | Concentration | Brand | |
|---|---|---|---|
| Reagent | Range (wt. %) | or Common Name | Vendor Name |
| Polyhexamethylene | 0.01–20 | Cosmocil CQ | Arch |
| biguanide (PHMB) | | | Chemicals, Inc. |
| | | | Norwalk, CT |
| Chitosan glycolate | 0.01–10 | Hydagen | Cognis Corp., |
| | | CMF and | Ambler, PA |
| | | HCMF | |
| Octadecylaminodimethyl | 0.01–10 | AEGIS AEM | Dow-Corning, |
| Trimethoxysilylpropyl | | 5700 | Midland, MI |
| Ammonium Chloride | | | |
| N-Alkyl Polyglycoside | 0.01–10 | Glucopon 220 | Cognis Corp., |
| | | UP | Ambler, PA |
| PG-Hydroxyethylcellulose | 0.01–10 | Crodacel QM | Croda Inc., |
| Cocodimonium Chloride | | | Persipanny, NJ |
| (Quaternary Ammonium | | | |
| CellulosicSalt) | | | |
| Xylitol | 0.01–10 | Xylitol | Sigma-Aldrich, |
| | | | Milwaukee, WI |
| 2-hydroxy-1,2,3- | 0.01–10 | Citric Acid | Hach |
| propanetricarboxylic acid | | | Company |
| | | | Ames, IA |
| Benzenecarboxylic acid | 0.1–2.0 | Benzoic acid | Mallinckrodt |
| | | | Baker, Inc |
| | | | Phillipsburg, NJ |
| 2-hydroxybenzoic acid | 0.01–10 | Salicylic acid | Mallinckrodt |
| | | | Baker, Inc |
| | | | Phillipsburg, NJ |
| Methane-carboxylic acid | 0.01–2.0 | Acetic acid | Sigma-Aldrich |
| | | | St. Louis, MO |
| 1,3-Propanedicarboxylic | 0.01–10 | Glutaric acid | Sigma-Aldrich |
| Acid | | | St. Louis, MO |
| Iodine | 0.05–10 | Iodine | Sigma-Aldrich |
| | | | St. Louis, MO |
| Ethyl Hydroxyethyl cellulose | 0.01–5.0 | Bermocoll | Akzo Nobel, |
| | | EBS 481 FQ | Inc., |
| | | (“E 481”) | Stamford, CT |
| Polyvinyl pyrrolidone | 0.01–10 | Plasdone K90 | ISP |
| | | | Technologies, |
| | | | Inc., |
| | | | Wayne, NJ |
| Poly(vinyl pyrrolidone-co- | 0.01–10 | PVP/VA S- | ISP |
| vinyl acetate) | | 630 | Technologies, |
| | | | Inc., |
| | | | Wayne, NJ |
| Polyvinyl pyrrolidone-Iodine | 0.01–10 | PVP-Iodine | ISP |
| complex | | | Technologies, |
| | | | Inc., |
| | | | Wayne, NJ |
| Guanidine Hydrochloride | 0.01–5.0 | Nicepole FL | NICCA USA, |
| and Sorbitol | | | Inc. |
| | | | Fountain Inn, |
| | | | SC |
| Acrylic Co-Polymer | 0.01–5.0 | Nicepole FE | NICCA U.S.A., |
| Compound and Isopropyl | | 18U | Inc. |
| Alcohol | | | Fountain Inn, |
| | | | SC |
| 25% Copper oxide (CuO, | 0.01–20.0 | Cupron* | Cupron, Inc. |
| Cu₂O) (CAS #1317-39-1), | | | Greensboro, |
| 75% polypropylene (PP) | | | NC |
| resin | | | |
| Silver Sodium Hydrogen | 0.01–20.0 | AlphaSan ® RC | Milliken, |
| Zirconium Phosphate | | 2000* | Spartanburg, |
| | | | SC |
| Silver Zinc glass (70–100%,) | 0.01–20.0 | Irgaguard B | Ciba Specialty |
| barium sulfate (1–30%), PP | | 7520* | Chemicals |
| resin (10–30%) | | | Corp. |
| | | | Tarrytown, NY |

*Used as internal melt additives. These additives are typically compounded in thermoplastic resins (e.g., polypropylene (PP)) to produce a concentrate which is then dry blended with the virgin resin and co-extruded to produce fibers and webs containing such additives. The additive is generally distributed throughout the bulk of the fiber and enough of the additive is present on the surface of the fiber to provide anti-microbial activity. Concentration of the additive present on the surface of the fiber depends on several factors including additive concentration in the melt relative to the main body of resin or type of resin, processing conditions and thermal history, crystallinity of the resin, and relative thermodynamic compatibility of the resin and the additive. It is understood that the additive must be compatible with thermoplastic resin in the melt for processability, and yet it is desirable that the additive be less compatible with the resin at ambient conditions so that the additive migrate to a certain extent to the surface of the thermoplastic fiber. Processing aids such as amorphous compounds can be added to the main resin to ease migration of the additive to the fiber surface. It is also understood that other active ingredients such as PHMB can be compounded and co-extruded in various other thermoplastic resins.

Referring again to FIGS. 1-3, the drape may include an opening 120 that is placed over the surgical site such that the surgical procedure is performed through the opening 120. The opening can have any desired shape and dimensions. Desirably, the opening is occluded or partially occluded by a connecting element 50.

The central region 60 may be incorporated into the base sheet 80 around the opening 120. This central region may surround the opening 120, as illustrated in the figures, or be disposed along one or more sides of the opening 20. The central region may be formed of the same material as the base sheet 80, may be formed of a different material than the base sheet, or may incorporate the material of the base sheet with a different material. For example, the central region may be an absorbent multi-layered nonwoven fabric. One or more of the layers may be a film. The base sheet may be hydrophilic or hydrophobic, and may be chemically treated to achieve a desired absorbency property. The central region may be a spunbond layer attached to a middle layer of a meltblown material, which is further attached to a backing layer of impervious film. This configuration allows for the reinforcement of the areas around the opening, provides fluid absorption, and ensures a fluid impervious barrier. Additionally, the central region may be connected to the base sheet at a seam or it may be a seamless connection. In this regard, the seam or seamless connection is located where the base sheet transitions into the central region at one of more transition points 240 on the base sheet. In addition to utilization of a seam or seamless connection, the central region may be connected to the base sheet utilizing a fastening means. Non-limiting examples of fastening means include a hook and loop configuration which is described in U.S. Pat. No. 6,055,987 which is hereby incorporated by reference in its entirely. In this regard, a hook material may be located at the edge of the central region at the transition point 240 and the loop material may be located on the base sheet at the transition point. Of course, it is likewise contemplated that the loop material be located at the edge of the central region at the transition point 240 and the hook material be located on the base sheet at the transition point.

Desirably the central region may have a variety of shapes or configurations to accommodate a surgical or medical procedure instrument. These shapes or configurations include, but are not limited to a tube-like shapes, sock-like shapes, elongated pillowcases, cylindrical channels, rectangles or squares.

Desirably, the central region of the drape is formed from one or more flexible, softer materials that are easier to manipulate than the material of the base sheet. In this regard, the softer, easier to manipulate material grants better tactile sensation of an item surrounded by the central region such as endotracheal tube 30. Of course, it is contemplated that less flexible materials may be used. Non-limiting examples of the material appropriate for the central region include plastic film. Although not required, the plastic film should desirably be transparent.

The drape includes a connecting element 50 integrated into the central region 60 of the base sheet 80. Although a male fit 100 is illustrated, the connecting element 50 may enable a male or female fit into connector 40 of a medical device, such as an endotracheal tube 30.

It is contemplated that any medical device involving a tube or tube-like structure, which extends outside the body is appropriate for use with the surgical drape of the present invention. Non-limiting examples of medical devices include a bronchoscope, endotracheal tube, tracheostomy tube, vascular catheter, and urological catheter.

In FIG. 1 the connector 40 of the endotracheal tube 30 is connected into the connecting element 50 which is integrated into the central region 60. Upon connection, the central region completely surrounds the endotracheal tube 30. This prevents the endotracheal tube 30 from acquiring pathogens from the outside environment on its surface and it prevents pathogens already on the surface of the endotracheal tube from spreading onto the hands of the medical professional and further contaminating the medical or surgical site. This covering of the medical device is important because often during medical procedures the medical professional must manipulate the position of the medical device. This manipulation creates opportunities for pathogens to pass from medical device to medical professional or vice-versa.

Desirably, upon connection of the medical device into the connecting element, the medical professional will be able to manipulate the medical device without touching the medical device. In this regard, when performing a medical procedure, the medical professional will only touch the upper surface of the central region 220 and will not touch the surface of the medical device itself. Once the medical professional secures the outer surface of the central region with his hand, the lower surface of the central region 230 will be in communication with the medical device.

Thus, upon connection of the medical device into the connecting element, the lower surface of the central region 230 and, the upper surface of the central region 220 separate the medical professional's hands from the medical device. This prevents the transmission of pathogens from device to professional and vice-versa. Additionally, it prevents the transmission of airborne pathogens onto the surface of the medical device. This is important because if airborne pathogens are present on the surface of the device, they can travel down the device into the patient's body at the surgical or medical procedure site.

The connecting element is generally of standard size. Standard size generally ranges from 10 mm to 20 mm, more desirably 15 mm. However, size may vary widely depending upon the device to be connected into the connecting element. Thus, the connecting element and opening may be sized accordingly, to accommodate the type of medical device needed for the medical procedure.

As illustrated in FIGS. 2 and 3, the connecting element may optionally include an outside female port 90 located above the connecting element or outside male port located 190 above the connecting element. The female port 90 and/or male port enable the connecting of additional medical devices without removal of the drape. This maintains the sterile field created by the central region. For example, a Y-connector may be connected or ventilator may be reconnected to an endotracheal tube without compromising the sterile field by touching the endotracheal tube or lifting or moving the drape.

Like the connecting element, the outside female and male ports are generally of standard size. Standard size generally ranges from 10 mm to 20 mm, more desirably 15 mm. However, size may vary widely depending upon the device to be connected into the outside port. Thus, the outside port may be sized accordingly, to accommodate the type of medical device needed for the medical procedure.

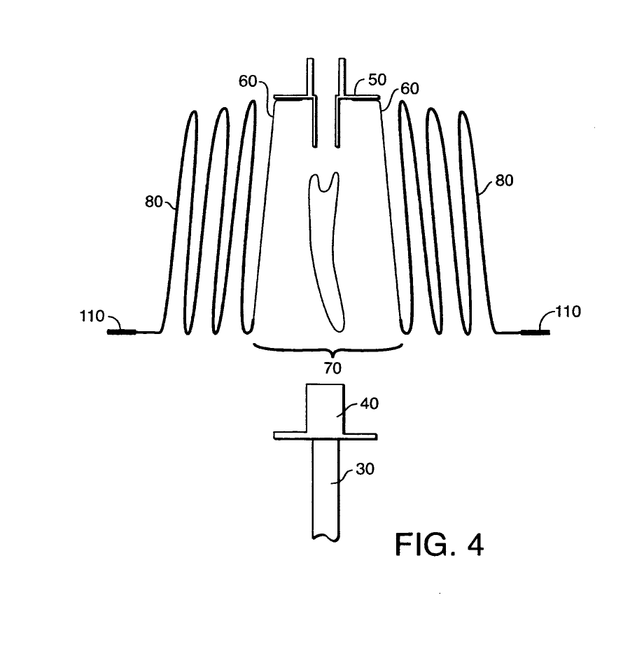

As illustrated in FIG. 4, the tracheal tube may be pre-shaped or pre-folded to form a central duct 70 defining the central region 60 to take up (to be put over) the shaft of the tracheal tube or other tubular medical device at the medical or surgical procedure site, when connecting it into the connector.

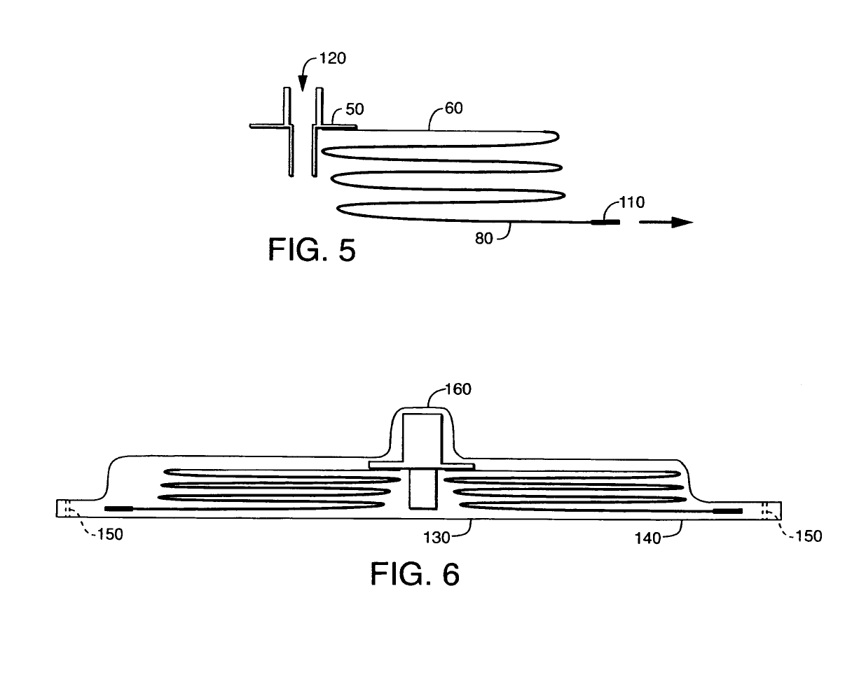

As illustrated in FIG. 5, the drape may be folded in such a manner as when a medical professional pulls the corner ends 110 of the drape it extends over the patient and the surgical site. The unfolded drape may extend partially over the patient's body or fully over the patient's body depending upon the type of surgical or medical procedure to be performed. Generally speaking, the drape may be pre-folded into any shape that allows the connecting element of the central region to be placed into the connector of a medical device without unfolding the drape, and that allows the drape to be thereafter unfolded by pulling on its corner ends 110, but the drape is desirably folded into an accordion-like shape.

Referring to FIG. 6, the pre-folded drape may be placed into a storage or shipping package 130. The package may be made of any material, but is desirably a flexible material such as, for example, plastic. The package may include a bottom end 140 and a top end 160 which collectively enclose the pre-folded drape and come into communication with each other at one or more enclosure ends 150. These enclosure ends may include any material that allows the bottom end 140 and top end 160 of the package to enclose the pre-folded drape until one end is removed by force. Desirably, the bottom end 140 and top end 160 are held together at the enclosure ends 150 by inclusion of a perforation or an adhesive at the enclosure end.

Prior to a surgical procedure, such as for example, a tracheal procedure, a tracheal tube may be inserted into the patient's throat. The bottom end 140 of the package may be peeled off to expose the connecting element and the connecting element may then be placed over the connector of the tracheal tube. The top end 160 may thereafter by removed and the corner ends 110 of the pre-folded drape may then be pulled laterally to extend over the patients body. Of course, the drape may be placed over the patient utilizing any sequence that allows the central region of the drape to shield the medical or surgical instrument from the outside atmosphere and allows the medical professional to manipulate the medical or surgical device without touching it.

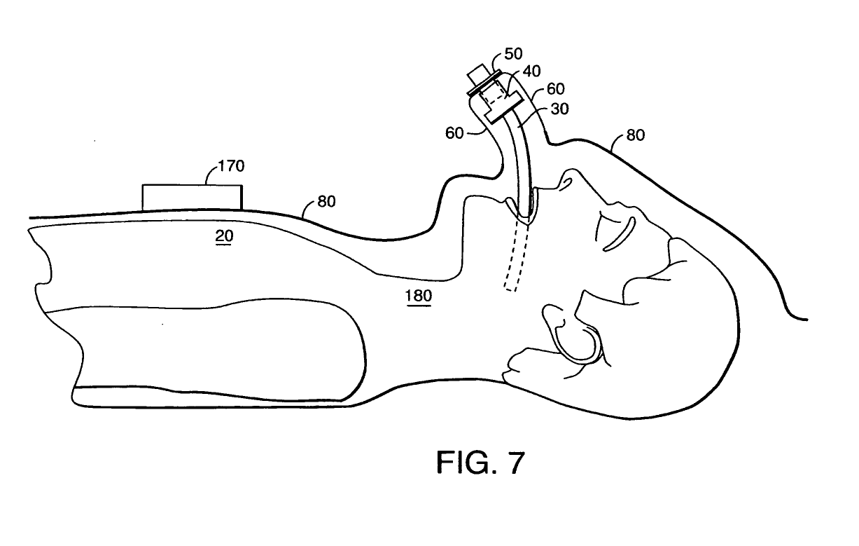

Referring to FIG. 7, it is also contemplated that a tray 170 may be used to enclose the pre-folded drape. The pre-folded drape may be removed from the tray and then used to cover the patient. The tray may then be placed on the patient's body and may contain materials for utilization during the surgical procedure, such as, for example syringes and cleaning swabs.

1. A drape for covering a patient during a medical procedure, the drape comprising:
a base sheet having an upper surface, a lower surface, a plurality of edges, and a central region defining an opening, the opening adapted to be positioned over the medical procedure site; and
a connecting element adapted to enable a male or female fit into a connector of a medical device, whereby the connecting element is in communication with the drape and is further adapted to occlude the opening.

2. The drape of claim 1, wherein the connecting element partially extends above the upper surface of the central region and partially extends below the lower surface of the central region.

3. The drape of claim 1, wherein at least a portion of the base sheet further comprises a layer of liquid absorbent material.

4. The drape of claim 1, wherein at least a portion of the base sheet further comprises a barrier fabric.

5. The drape of claim 1, wherein the central region of the base sheet defining the opening is adapted to surround the medical device upon insertion of the connecting element into the connector of the medical device and is further adapted to allow manipulation of the medical device without touching of the medical device.

6. The drape of claim 5, wherein the central region of the drape defining the opening comprises a plastic film.

7. The drape of claim 6, wherein the plastic film is transparent.

8. The drape of claim 1, wherein the connecting element comprises a female port.

9. The drape of claim 8, wherein the female port extends above the upper surface of the base sheet.

10. The drape of claim 8, wherein the female port has a diameter between about 10 and about 20 mm.

11. The drape of claim 1, wherein the connecting element is adapted to enable a male fit into a connector having a diameter between about 10 and about 20 mm.

12. The drape of claim 1, wherein the medical device is an endotracheal tube, tracheostomy tube, vascular catheter, bronchoscope, or urological catheter.

13. The drape of claim 1, wherein the drape is pre-folded so that the connecting element is available for connection with the connector.

14. The drape of claim 13, wherein the central region of the base sheet defining the opening is further defined by a central duct when the drape is pre-folded, the duct being adapted to receive the medical device and the connector.

15. The drape of claim 14, wherein the central region of the base sheet defining the opening is adapted to surround the medical device upon insertion of the connecting element into the connector of the medical device and is further adapted to allow manipulation of the medical device without touching of the medical device.

16. The drape of claim 1, wherein the drape further comprises a tray in communication with the base sheet.

17. The drape of claim 1, wherein the drape further comprises an outside male port.

18. The drape of claim 17, where in the outside male port extends above the upper surface of the base sheet.

19. The drape of claim 18, wherein the outside male port has a diameter between about 10 and about 15 mm.

20. A method of providing a sterile field about a patient during a medical procedure, the method comprising:
providing a drape comprising a base sheet having a connecting element which occludes an opening in the base sheet, the base sheet having an upper surface, a lower surface, and a plurality of edges;
positioning the opening over a medical procedure site;
connecting a connecting element into the connector of a medical device; and
manipulating the medical device without touching the medical device.

21. The method of claim 20, wherein the medical device is manipulated without touching the medical device by the steps of:
surrounding the medical device with a portion of the base sheet;
and securing the base sheet and medical device in a manner that causes the base sheet to have contact with the medical device but that does not allow for direct human contact with the medical device.

22. The method of claim 21, wherein the portion of the drape that surrounds the medical device comprises a transparent plastic film.

23. The method of claim 20, wherein the base sheet comprises a liquid absorbent material.

24. The method of claim 20, wherein the base sheet comprises a barrier fabric.

25. The method of claim 20, wherein the medical device is an endotracheal tube, tracheostomy tube, vascular catheter, bronchoscope, or urological catheter.

26. A drape adapted for covering a patient during a tracheal, bronchoscopic, vascular, or urological procedure, the drape comprising:
a base sheet having an upper surface, a lower surface, a plurality of edges, and a central region defining a opening, the opening adapted to be positioned over the tracheal or endoscopic procedure site; and
a connecting element adapted to enable a male or female fit into a connector of a medical device, whereby the connecting element is in communication with the drape and is further adapted to occlude the opening.
